(19) United States
(12) Patent Application Publication
(10) Pub. No.: US 2004/0234489 A1
(43) Pub. Date: Nov. 25, 2004

(54) Use of a particular carboxylic acid or salts thereof as agents for conditioning keratin materials

(75) Inventor: Rainer Muller, Leopoldshafen (DE)

Correspondence Address: LERNER, DAVID, LITTENBERG,, KRUMHOLZ & MENTLIK, 600 SOUTH AVENUE WEST, WESTFIELD, NJ, 07090, US

(73) Assignee: L'OREAL S.A., Paris (FR)

(21) Appl. No.: 10/807,149

(22) Filed: Mar. 23, 2004

Related U.S. Application Data
(60) Provisional application No. 60/461,213, filed on Apr. 8, 2003.
(60) Provisional application No. 60/461,218, filed on Apr. 8, 2003.

(30) Foreign Application Priority Data
Mar. 28, 2003 (FR) 0303879
Mar. 25, 2003 (FR) 0303637
Mar. 25, 2003 (FR) 0303641

(51) Int. Cl. A61K 7/075; A61K 7/08

(52) U.S. Cl. 424/70.22

(57) ABSTRACT

The invention relates to a method of conditioning a keratinous material comprising applying a composition comprising at least one carboxylic acid or a salt thereof corresponding to the formula (I) or (II):

R¹—(CHOH)₄—CO₂X (I)

R²—N—(CH(R′)COOX)₂ (II)

wherein:

R¹ represents a CH₂OH or CO₂X group,

X represents a hydrogen atom or a monovalent or divalent cation derived from a transition metal, alkali metal, alkaline-earth metal, organic amine or an ammonium ion,

R² represents a hydrogen atom or a —CH(COOX)—(CH₂)₂COOX, —CH₂CH₂OH, —CH(CH₃)—COOX or —(CH₂)₂—N(COR″)—CH₂—COOX group,

R″ represents a linear or branched alkyl group containing from 1 to 30 carbon atoms or a cyclic alkyl group containing from 3 to 30 carbon atoms, and

R′ represents either a —CH₂—COOX group when R² is a hydrogen atom, or a hydrogen atom when R² is other than a hydrogen atom

to the keratinous material.

### CROSS-REFERENCE TO RELATED APPLICATIONS

The present application claims the benefit of French Application No. 0303637, filed Mar. 25, 2003, French Application No. 0303641, filed Mar. 25, 2003, French Application No. 0303879, filed Mar. 28, 2003, U.S. Provisional Application No. 60/461,218, filed Apr. 8, 2003, and U.S. Provisional Application No. 60/461,213, filed Apr. 8, 2003, the disclosures of which are incorporated by reference herein.

The present invention relates to the method of using a particular carboxylic acid or a salt thereof, as an agent for conditioning a keratinous material, in and for the preparation of cosmetic compositions, as agents for conditioning a keratin material, in particular keratin fibers and more preferably the hair.

### BACKGROUND OF THE INVENTION

Cosmetic compositions generally contain a complexing agent intended to complex the metal cations that may be present in trace amounts in these compositions, and also those that may be present on the hair, originating from the ambient air, from the water with which this hair has been washed, or shampoos or other hair products with which the hair has been treated.

Specifically, it is very important to neutralize these metal cations, since they are capable of catalysing oxidation reactions on the hair fibers, and of doing so in an uncontrolled manner, which be reflected by severe adverse effects such as breaking of the hair or burning of the scalp.

The complexing agents that are currently the most commonly used in oxidizing compositions for dyeing, bleaching or permanently reshaping keratin fibers are ethylenediaminetetraacetic acid (EDTA) and derivatives thereof, for instance diethylenetriaminepentaacetic acid (DPTA), generally in weight proportions of about 0.1% to 1%. These complexing agents are not sufficiently biodegradable.

Moreover, the Applicant has found that EDTA and derivatives thereof have insufficient complexing properties in compositions of this type. These findings, which are corroborated by the results obtained by other research teams, justify the search for novel complexing agents.

### SUMMARY OF THE INVENTION

The Applicant has discovered, surprisingly, that particular complexing agents, used in cosmetic compositions, preferably hair compositions, give the hair a particularly soft feel and facilitate its disentangling. They can thus be used as agents for conditioning keratin materials, preferably keratin fibers and more preferably the hair.

One preferred embodiment of the invention relates to a method of using at least one particular polycarboxylic acid or a salt thereof, as an agent for conditioning a keratin material, in particular keratin fibers and more preferably the hair, in and for the preparation of cosmetic compositions, such as hair compositions.

Other preferred embodiment will become apparent in light of the description and the examples that follow.

### DETAILED DESCRIPTION OF THE INVENTION

In the context of the present patent application, the term “conditioning agent” means an agent whose function is to improve the cosmetic properties of the hair, preferably the softness, the disentangling, the feel, or sheen and the static electricity.

The particular carboxylic acids or salts thereof are chosen from the acids of general formula (I) or (II) below:

R¹—(CHOH)₄—CO₂X (I)

R²—N—(CH(R′)COOX)₂ (II)

in which:

R¹ represents a CH₂OH or CO₂X group,

X represents a hydrogen atom or a monovalent or divalent cation derived from a transition metal, from an alkali metal or alkaline-earth metal, from an organic amine or from an ammonium ion,

R² represents a hydrogen atom or a group —CH(COOX)—(CH₂)₂COOX, —CH₂CH₂OH, —CH(CH₃)—COOX or —(CH₂)₂—N(COR″)—CH₂—COOX;

R″ represents a linear or branched alkyl group containing from 1 to 30 carbon atoms or a cyclic alkyl group containing from 3 to 30 carbon atoms;

R′ represents a group —CH₂—COOX when R² represents a hydrogen atom, whereas R′ represents a hydrogen atom when R² is other than a hydrogen atom.

Thus, the complexing agent of formula (I) used in the context of the invention corresponds to hydroxycarboxylic acid and to the corresponding carboxylate.

Since formula (I) comprises 4 chiral groups of H—C—OH atoms, and even 5 when R¹ represents a CH₂OH group, it is known to the skilled in the art that this formula includes all the enantiomers, diastereoisomers, racemic mixtures and other mixtures of which would satisfy this formula.

In accordance with the invention, when the compound(s) of formula (I) is(are) carboxylate(s), then the monovalent or divalent cation is preferably chosen from the group consisting of alkali metal cations, alkaline-earth metal cations, divalent transition metal cations and monovalent cations derived from organic amines or from ammonium.

Examples of alkali metal cations that may be mentioned include sodium (Na⁺) and potassium (K⁺), while examples of alkaline-earth metal cations that may be mentioned include calcium (Ca²⁺) and magnesium (Mg²⁺)

For the purpose of the present invention, the term “transition metal” means a metal comprising an incomplete d subshell, more preferably in oxidation state II, such as cobalt (Co²+), iron (Fe²⁺), manganese (Mn²⁺), zinc (Zn²⁺) and copper (Cu²⁺)

With regard to the organic amine salts, mention may be made of primary, secondary or tertiary amine salts or alternatively alkanolamine salts.

The said amines contain one or more radicals, which may be identical or different, of linear or branched C1 to C20 alkyl type, optionally comprising a hetero atom such as oxygen.

With regard to the quaternary ammonium salts, these comprise three radicals, which may be identical or different, chosen from hydrogen and a linear or branched C1 to C20 alkyl radical, optionally comprising a hetero atom such as oxygen.

When R¹ represents a CH₂OH group, then the compound(s) of formula (I) is(are) preferably chosen from the group consisting of gluconic acid (C₆H₁₂O₇), the alkali metal salts thereof, the alkaline-earth metal salts thereof, the transition metal salts thereof, and mixtures thereof, for instance mixtures of gluconic acid and of sodium gluconate.

More preferably, the compound(s) of formula (I) is(are) then chosen from the group consisting of gluconic acid, sodium gluconate (C₆H₁₁O₇Na), potassium gluconate (C₆H₁₁O₇K), anhydrous calcium gluconate (C₁₂H₂₂O₁₄Ca), calcium gluconate monohydrate (C₁₂H₂₂O₁₄Ca.H₂O), calcium borogluconate (C₁₂H₂₂O₁₄Ca.H₂O+H₅BO₅), magnesium gluconate (C₁₂H₂₂O₁₄Mg), iron gluconate (C₁₂H₂₂O₁₄Fe), manganese gluconate (C₁₂H₂₂O₁₄Mn), zinc gluconate (C₁₂H₂₂O₁₄Zn) and copper gluconate (C₁₂H₂₂O₁₄Cu).

When R¹ represents a group CO₂X, then the compound(s) of formula (I) is(are) preferably chosen from the group consisting of mucic acid (C₆H₁₀O₈)— also known as galactaric acid—, glucaric acid (C₆H₁₀O₈) and mannaric acid (C₆H₁₀O₈), the alkali metal salts thereof, the alkaline-earth metal salts thereof, the transition metal salts thereof, and mixtures thereof, for example mixtures of mucic acid and of sodium mucate (C₆H₈O₈Na₂).

In a particularly preferred embodiment, the compound(s) of formula (I) is(are) chosen from gluconic acid and mucic acid.

Thus, the acid of formula (II) used in the context of the invention correspond to a polycarboxylic acid compound and to the corresponding carboxylate.

More preferably, they correspond to:

a compound comprising four carboxylic acid or carboxylate functions, when R² represents a hydrogen atom and R′ represents a group —CH₂—COOX, or when R² represents a group —CH(COOX)—(CH₂)₂—COOX and R′ represents a hydrogen atom;

a compound comprising three carboxylic acid or carboxylate functions, when R² represents a group —CH(CH₃)—COOX and R′ represents a hydrogen atom, or when R² represents a group —(CH₂)₂—N(COR″)—CH₂—COOX and R′ represents a hydrogen atom; and

a compound comprising two carboxylic acid or carboxylate functions, when R² represents a —CH₂CH₂OH group and R′ represents a hydrogen atom.

The compound(s) of formula (I) is(are) preferably chosen from the group consisting of methylglycinediacetic acid, N-lauroyl-N,N′,N′-ethylenediaminetriacetic acid, N,N-dicarboxymethyl-L-glutamic acid and iminodisuccinic acid, the alkali metal salts thereof, the alkaline-earth metal salts thereof, the transition metal salts thereof, the organic amine salts thereof or the ammonium salts thereof, or mixtures thereof.

Methylglycinediacetic acid, 2-hydroxyethyliminodiacetic acid, N-lauroyl-N,N′,N′-ethylenediaminetriacetic acid, N,N-dicarboxymethyl-L-glutamic acid and iminodisuccinic acid, and the salts thereof, are respectively represented by formulae (III), (IV), (V), (VI) and (VII) below:

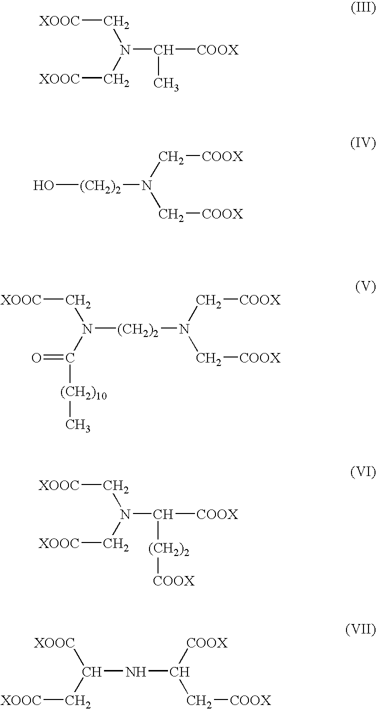

in which X is as defined above, X preferably corresponding to H or Na.

These compounds are available from the companies BASF, Dow Chemical, Hampshire, Bayer and Showa Denko.

Methylglycinediacetic acid and the sodium salts thereof are more particularly preferred.

Preferably, the compound(s) of formula (I) or of formula (II) represent(s) from 0.001% to 10% by weight and more preferably from 0.001% to 5% by weight relative to the total weight of the composition.

The cosmetic compositions used according to the invention contain a cosmetically acceptable medium, i.e. a medium that is compatible with the skin, the lips, the scalp, the eyelashes, the eyes, the nails and/or the hair. This cosmetically acceptable medium may consist preferably of water and optionally of a physiologically acceptable organic solvent chosen, for example, from lower alcohols containing from 1 to 8 carbon atoms and preferably 1 to 6 carbon atoms, for instance ethanol, isopropanol, propanol or butanol; polyethylene glycols containing from 6 to 80 ethylene oxides; polyols, for instance propylene glycol, isoprene glycol, butylene glycol, glycerol or sorbitol.

The compositions may be in the form of a one-phase or multiphase aqueous or aqueous-alcoholic lotion, a one-phase or multiphase gel, an emulsion, a cream, a vesicular dispersion of ionic or nonionic lipids, the said vesicles then possibly serving as agents for encapsulating lipophilic or hydrophilic active ingredients, a mousse or a spray.

In particular, the skincare compositions according to the invention may be in the form of a lotion, a gel, an emulsion, a cream or a mousse to be applied to the skin.

The hair compositions may be in the form of a shampoo, a rinse-out or leave-in conditioning agent, compositions for permanent-waving, relaxing, dyeing or bleaching the hair, or alternatively in the form of rinse-out compositions, to be applied before or after dyeing, permanent-waving or relaxing the hair or between the two steps of a permanent-waving or hair-relaxing operation.

The cosmetic or dermatological compositions may moreover contain conventional cosmetic additives chosen from fatty substances, organic solvents, silicones, thickeners, softeners, surfactants, anionic, cationic, nonionic or amphoteric polymers, antifoams, hair conditioning agents such as proteins, vitamins, treatment agents (agents for preventing hair loss or antidandruff agents), dyes, fragrances, preserving agents and propellants.

Among the oils that may be mentioned are mineral oils, animal oils, plant oils or synthetic oils, and preferably liquid petroleum jelly, liquid paraffin, castor oil, jojoba oil, sesame seed oil, silicone oils and gums, and isoparaffins.

Among the waxes that may be mentioned are animal waxes, plant waxes, mineral waxes or synthetic waxes, and preferably beeswax, candelilla wax, ozokerites, microcrystalline waxes and silicone waxes and resins.

Among the organic solvents usually used in cosmetic compositions, mention may be made more specifically of C₁ to C₆ lower monoalcohols or polyalcohols, for instance ethanol, isopropanol, ethylene glycol, diethylene glycol, propylene glycol and glycerol.

The thickeners may be chosen preferably from sodium alginate, gum arabic, cellulose derivatives such as methylcellulose, hydroxymethylcellulose, hydroxyethylcellulose, hydroxypropylcellulose and hydroxypropylmethylcellulose, guar gum or derivatives thereof, xanthan gum, scleroglucans, crosslinked polyacrylic acids and associative polymers, preferably fatty-chain (C6-C30) polymers.

Surfactants and polymers that may be used include any of those that well known to the skilled in the art for their use in hair compositions.

The polymers, for example the cationic polymers, that may be used in the context of the present invention are preferably quaternary cellulose ether derivatives such as the products sold under the name “JR 400” by the company Amerchol, cationic cyclopolymers, in particular the dimethyldiallylammonium chloride homopolymers or copolymers sold under the names “Merquat 100”, “Merquat 550” and “Merquat S” by the company Nalco, and quaternary polymers of vinylpyrrolidone and of vinylimidazole, and mixtures thereof.

Several examples of cosmetic compositions will now be given by way of illustration and with no limiting nature.

### EXAMPLE 1

The following shampoo compositions are prepared:

| | A invention | B comparative |
|---|---|---|
| | in g | in g |
| Sodium lauryl ether | 12 | 12 |
| (2 EO) sulphate (70% AM) | | |
| Cocobetaine (32% AM) | 10 | 10 |
| Coconut monoethanolamide | 0.50 | 0.50 |
| (Cocamide MEA) | | |
| Laureth-12 | 0.25 | 0.25 |
| Mucic acid (galactaric | 0.30 | — |
| acid)* | | |
| Dye | qs | qs |
| Fragrance | 0.50 | 0.50 |
| Preserving agent | 0.40 | 0.40 |
| Sodium hydroxide qs | pH 6.7 | pH 6.7 |
| Hexylene glycol | qs | qs |
| Water qs | 100 | 100 |

*Muciliance from the company Soliance

The two formulations are compared, per half-head, on 16 models. Moistened hair treated with the composition according to the invention has a smoother feel. The dried hair is softer and smoother.

### EXAMPLE 2

The following shampoo composition is prepared:

| | in g |
|---|---|
| Sodium lauryl ether | 12 |
| (2 EO) sulphate (70% AM) | |
| Cocobetaine (32% AM) | 10 |
| Coconut monoethanolamide | 0.50 |
| (Cocamide MEA) | |
| Laureth-12 | 0.25 |
| Methylglycinediacetic | 0.30 |
| acid, in trisodium salt | |
| form, as an aqueous 40% | |
| solution (Trilon M | |
| Liquid ® from the company | |
| BASF) | |
| Dye | 0.01 |
| Fragrance | 0.50 |
| Preserving agent | 0.40 |
| Sodium hydroxide qs | pH 6.7 |
| Hexylene glycol | 0.5 |
| Water qs | 100 |

### EXAMPLE 3

A shampoo composition is prepared:

| | in g |
|---|---|
| Guar | 0.05 |
| hydroxypropyltrimonium | |
| chloride | |
| Cocobetaine (32% AM) | 9 |
| Sodium lauryl ether | 22.2 |
| (2 EO) sulphate (70% AM) | |
| Sodium methyl paraben | 0.2 |
| DMDM hydantoin | 0.25 |
| Mucic acid (galactaric | 0.3 |
| acid) | |
| Dimethicone (DC 200 Fluid | 2.7 |
| 300 000 from Dow Corning) | |
| Mixture of cetyl alcohol | 2.5 |
| and of 1-(hexadecyloxy)- | |
| 2-octadecanol | |
| Fragrance | 0.5 |
| Coconut | 0.3 |
| monoisopropanolamide | |
| (Cocamide MIPA) | |
| Carbomer | 0.2 |
| Sodium hydroxide qs | pH 7 |
| Water qs | 100 |

Hair treated with this shampoo is soft and manageable.

1. A method of conditioning a keratinous material comprising:
applying a composition comprising at least one carboxylic acid or a salt thereof chosen from the compound of formula (I) or (II):
R¹—(CHOH)₄—CO₂X (I)
R²—N—(CH(R′)COOX)₂ (II),
wherein
R¹ represents a CH₂OH or CO₂X group,
X represents a hydrogen atom or a monovalent or divalent cation derived from a transition metal, alkali metal, alkaline-earth metal, organic amine or ammonium ion,
R² represents a hydrogen atom or a —CH(COOX)—(CH₂)₂COOX, —CH₂CH₂OH, —CH(CH₃)—COOX or —(CH₂)₂—N(COR″)—CH₂—COOX group,
R″ represents a linear or branched alkyl group containing from 1 to 30 carbon atoms or a cyclic alkyl group containing from 3 to 30 carbon atoms, and
R′ represents either a —CH₂—COOX group when R² is a hydrogen atom or a hydrogen atom when R² is other than a hydrogen atom,
to said keratinous material.

2. The method of claim 1, wherein said keratinous material is a keratinous fiber.

3. The method of claim 2, wherein said keratinous material is hair.

4. The method of claim 1, wherein said monovalent or said divalent cation is an alkali metal cation, an alkaline-earth metal cation or a divalent transition metal cation.

5. The method of claim 1, wherein said compound of formula (I) is gluconic acid, an alkali metal salt thereof, an alkaline-earth metal salt thereof, a transition metal salt thereof, or a mixture thereof.

6. The method of claim 5, wherein said compound of formula (I) is gluconic acid, sodium gluconate, potassium gluconate, anhydrous calcium gluconate, calcium gluconate monohydrate, calcium borogluconate, magnesium gluconate, iron gluconate, manganese gluconate, zinc gluconate or copper gluconate.

7. The method of claim 1, wherein when R¹ is a CH₂OH group, said compound of formula (I) is gluconic acid, an alkali metal salt thereof, an alkaline-earth metal salt thereof, a transition metal salt thereof, or a mixture thereof.

8. The method of claim 1, wherein said compound of formula (I) is mucic acid, glucaric acid, mannaric acid, or an alkali metal salt thereof, an alkaline-earth metal salt thereof, a transition metal salt thereof, or a mixture thereof.

9. The method of claim 1, wherein when R₁ is a CO₂X group, said compound of formula (I) is mucic acid, glucaric acid, mannaric acid, or an alkali metal salt thereof, an alkaline-earth metal salt thereof, a transition metal salt thereof, or a mixture thereof.

10. The method of claim 1, wherein said compound of formula (I) is gluconic acid or mucic acid.

11. The method of claim 1, wherein said compound of formula (II) is methylglycinediacetic acid, 2-hydroxyethyliminodiacetic acid, N-lauroyl-N,N′,N′-ethylenediaminetriacetic acid, iminodisuccinic acid, N,N-dicarboxymethyl-L-glutamic acid or the corresponding salt thereof or a mixture thereof.

12. The method of claim 11, wherein said compound of formula (II) is methylglycinediacetic acid, optionally in a salt form.

13. The method of claim 11, wherein said compound of formula (II) is 2-hydroxyethyliminodiacetic acid, optionally in a salt form.

14. The method of claim 11, wherein said compound of formula (II) is iminodisuccinic acid, optionally in a salt form.

15. The method of claim 1, wherein content of said compound of formula (I) or (II) is from 0.001% to 10% by weight relative to the total weight of the composition.

16. The method of claim 15, wherein said content of said compound of formula (I) or (II) is from 0.001% to 5% by weight relative to the total weight of the composition.

17. The method of claim 1, further comprising a cosmetic additive chosen from a fatty substance, organic solvent, silicone, thickener, softener, surfactant, anionic, cationic, nonionic or amphoteric polymer, antifoam, protein, vitamin, agent for preventing hair loss, antidandruff agent, dye, fragrance, preserving agent or propellant.

18. The method as in claim 1 or 17, wherein said composition is a shampoo, rinse-out or leave-in conditioning agent, composition for permanent-waving, relaxing, dyeing or bleaching the hair or rinse-out composition to be applied before or after dyeing, permanent-waving or relaxing the hair or between the two steps of permanent-waving or hair-relaxing operation.

19. The method of improving disentangling, softness or sheen of a keratinous material comprising:
applying a composition comprising at least one carboxylic acid or a salt thereof chosen from the compound of formula (I) or (II):
R¹—(CHOH)₄—CO₂X (I)
R²—N—(CH(R¹)COOX)₂ (II),
wherein
R¹ represents a CH₂OH or CO₂X group,
X represents a hydrogen atom or a monovalent or divalent cation derived from a transition metal, alkali metal, alkaline-earth metal, organic amine or from ammonium ion,
R² represents a hydrogen atom or a —CH(COOX)—(CH₂)₂COOX, —CH₂CH₂OH, —CH(CH₃)—COOX or —(CH₂)₂—N(COR″)—CH₂—COOX group,
R″ represents a linear or branched alkyl group containing from 1 to 30 carbon atoms or a cyclic alkyl group containing from 3 to 30 carbon atoms, and
R′ represents either a —CH₂—COOX group when R² is a hydrogen atom, or a hydrogen atom when R² is other than a hydrogen atom
to said keratinous material.

20. The method of claim 19, wherein said keratinous material is a keratinous fiber.

21. The method of claim 20, wherein said keratinous material is hair.

22. A method of conditioning a keratinous material comprising:
dispensing a composition comprising at least one carboxylic acid or a salt thereof chosen from the compound of formula (I) or (II):
R¹—(CHOH)₄—CO₂X (I)
R²_N—(CH(R′)COOX)₂ (II),
wherein
R¹ represents a CH₂OH or CO₂X group,
X represents a hydrogen atom or a monovalent or divalent cation derived from a transition metal, alkali metal, alkaline-earth metal, organic amine or ammonium ion,
R² represents a hydrogen atom or a —CH(COOX)—(CH₂)₂COOX, —CH₂CH₂OH, —CH(CH₃)—COOX or —(CH₂)₂—N(COR″)—CH₂—COOX group,
R″ represents a linear or branched alkyl group containing from 1 to 30 carbon atoms or a cyclic alkyl group containing from 3 to 30 carbon atoms, and
R′ represents either a —CH₂—COOX group when R² is a hydrogen atom or a hydrogen atom when R² is other than a hydrogen atom,
and then applying said composition to said keratinous material.

23. A method as in claim 1, 19 or 22, further comprising rinsing said composition from said keratinous material with water.

24. A product for application to hair comprising:
at least one carboxylic acid or a salt thereof chosen from the compound of formula (I) or (II):
R¹—(CHOH)₄—CO₂X (I)
R²—N—(CH(R′)COOX)₂ (II),
wherein
R¹ represents a CH₂OH or CO₂X group,
X represents a hydrogen atom or a monovalent or divalent cation derived from a transition metal, alkali metal, alkaline-earth metal, organic amine or from ammonium ion,
R² represents a hydrogen atom or a —CH(COOX)—(CH₂)₂COOX, —CH₂CH₂OH, —CH(CH₃)—COOX or —(CH₂)₂—N(COR″)—CH₂—COOX group;
R″ represents a linear or branched alkyl group containing from 1 to 30 carbon atoms or a cyclic alkyl group containing from 3 to 30 carbon atoms; and
R′ represents either a —CH₂—COOX group when R² is a hydrogen atom, or a hydrogen atom when R² is other than a hydrogen atom,
formulated as a shampoo, rinse-out or leave-in conditioning agent, composition for permanent-waving, relaxing, dyeing or bleaching the hair or rinse-out composition to be applied before or after dyeing, permanent-waving or relaxing the hair or between the two steps of permanent-waving or hair-relaxing operation.

25. The product for application for hair of claim 24, further comprising a cosmetic additive chosen from a fatty substance, organic solvent, silicone, thickener, softener, surfactant, anionic, cationic, nonionic or amphoteric polymer, antifoam, protein, vitamin, agent for preventing hair loss, antidandruff agent, dye, fragrance, preserving agent or properllant.
